(19) United States
(12) Patent Application Publication
(10) Pub. No.: US 2019/0388177 A1
(43) Pub. Date: Dec. 26, 2019

(54) SURGICAL NAVIGATION METHOD AND SYSTEM USING AUGMENTED REALITY

(71) Applicants: Chang Gung University, Taoyuan City (TW); Chang Gung Memorial Hospital, Linkou, Taoyuan City (TW)

(72) Inventors: Shin-Yan Chiou, Zhubei City (TW); Hao-Li Liu, Taoyuan City (TW); Chen-Yuan Liao, New Taipei City (TW); Pin-Yuan Chen, Taoyuan City (TW); Kuo-Chen Wei, Taoyuan City (TW)

(21) Appl. No.: 16/375,654

(22) Filed: Apr. 4, 2019

(30) Foreign Application Priority Data
Jun. 26, 2018 (TW) 107121828

(51) Int. Cl. A61B 90/00; G06T 19/00; G06T 11/60; A61B 5/06; A61B 34/20; A61B 34/00

(52) U.S. Cl. CPC A61B 90/37; G06T 19/006; G06T 11/60; A61B 5/066; A61B 5/067; A61B 2090/365; A61B 34/25; G06T 2210/41; A61B 2034/2055; A61B 2034/2048; A61B 2034/254; A61B 34/20

(57) ABSTRACT

In a proposed surgical navigation method for a surgical operation to be performed on an operation target, a mobile device stores 3D imaging information that relates to the operation target before the surgical operation. Then, an optical positioning system is used to acquire spatial coordinate information relating to the mobile device and the operation target, so that the mobile device can obtain a relative coordinate which is a vector from the operation target to the mobile device, obtain a 3D image based on the relative coordinate and the 3D imaging information, and display the 3D image based on the relative coordinate, such that visual perception of the operation target through the mobile device has the 3D image superimposed thereon.

### CROSS-REFERENCE TO RELATED APPLICATION

This application claims priority of Taiwanese Invention Patent Application No. 107121828, filed on Jun. 26, 2018, the entire teachings and disclosure of which is incorporated herein by reference.

### FIELD

The disclosure relates to a surgical navigation method, and more particularly to a surgical navigation method using augmented reality.

### BACKGROUND

Surgical navigation systems have been applied to neurosurgical operations for years in order to reduce damages to patients' bodies during the operations due to the intricate cranial nerves, narrow operating space, and limited anatomical information. The surgical navigation systems may help a surgeon locate a lesion more precisely and more safely, provide information on relative orientations of bodily structures, and serve as a tool for measuring distances or lengths of bodily structures, thereby aiding in the surgeon's decision making process during operations.

In addition, the surgical navigation systems may need to precisely align pre-operation data, such as computerized tomography (CT) image, magnetic resonance imaging (MRI) images, etc., with the head of the patient, such that the images are superimposed on the head in the surgeon's visual perception through a display device. Precision of the alignment is an influential factor in the precision of the operation.

### SUMMARY

Therefore, an object of the disclosure is to provide a surgical navigation method that can superimpose images on an operation target during a surgical operation with high precision.

According to the disclosure, the surgical navigation method includes, before the surgical operation is performed: (A) by a mobile device that is capable of computation and displaying images, storing three-dimensional (3D) imaging information that relates to the operation target therein; and includes, during the surgical operation: (B) by an optical positioning system, acquiring optically-positioned spatial coordinate information relating to the mobile device and the operation target in real time; (C) by the mobile device, obtaining a first optically-positioned relative coordinate set, which is a vector from the operation target to the mobile device, based on the optically-positioned spatial coordinate information acquired in step (B); and (D) by the mobile device, computing an optically-positioned 3D image that corresponds to the first optically-positioned relative coordinate set based on the 3D imaging information and the first optically-positioned relative coordinate set, and displaying the optically-positioned 3D image based on the first optically-positioned relative coordinate set such that visual perception of the operation target through the mobile device has the optically-positioned 3D image superimposed thereon.

Another object of the disclosure is to provide a surgical navigation system that includes a mobile device and an optical positioning system to implement the surgical navigation method of this disclosure.

### BRIEF DESCRIPTION OF THE DRAWINGS

Other features and advantages of the disclosure will become apparent in the following detailed description of the embodiment(s) with reference to the accompanying drawings, of which:

FIG. 1 is a flow chart illustrating a first embodiment of the surgical navigation method according to the disclosure;

FIG. 2 is a schematic diagram illustrating a surgical navigation system used to implement the first embodiment;

FIG. 3 is a schematic diagram illustrating another surgical system used to implement a second embodiment of the surgical navigation method according to the disclosure;

FIG. 4 is a flow chart illustrating the second embodiment;

FIG. 5 is a flow chart illustrating sub-steps of step S42 of the second embodiment;

FIG. 6 is a flow chart illustrating a third embodiment of the surgical navigation method according to the disclosure

FIG. 7 is a schematic diagram that exemplarily shows visual perception of an operation target through a mobile device of the surgical navigation system, the visual perception having several optically-positioned 2D images superimposed thereon; and

FIG. 8 is a schematic diagram that exemplarily shows visual perception of an operation target through a mobile device of the surgical navigation system, the visual perception having a 3D image superimposed thereon and two 2D images displayed aside.

### DETAILED DESCRIPTION

Before the disclosure is described in greater detail, it should be noted that where considered appropriate, reference numerals or terminal portions of reference numerals have been repeated among the figures to indicate corresponding or analogous elements, which may optionally have similar characteristics.

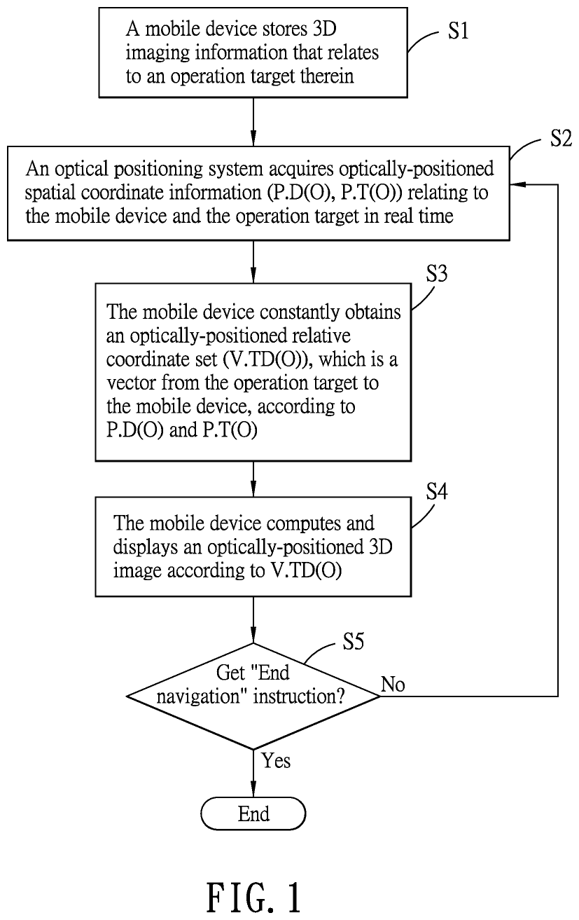

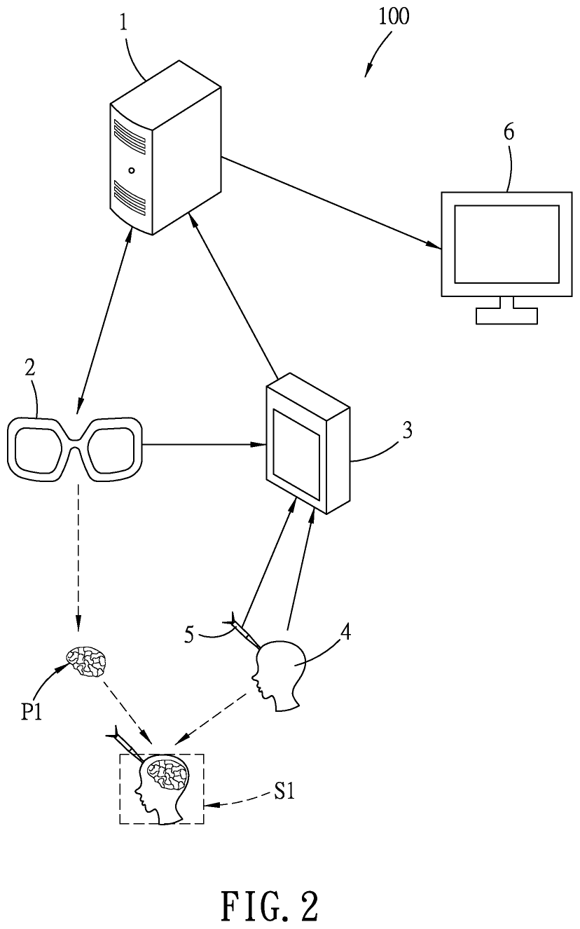

Referring to FIG. 1 and FIG. 2, the first embodiment of the surgical navigation method according to this disclosure is implemented by a surgical navigation system 100 that uses augmented reality (AR) technology. The surgical navigation system 100 is applied to surgical operation. In this embodiment, the surgical operation is exemplified as a brain surgery, but this disclosure is not limited in this respect. The surgical navigation system 100 includes a server 1, a mobile device 2 that is capable of computation and displaying images and that is for use by a surgeon and/or relevant personnel, and an optical positioning system 3. The server 1 is communicatively coupled to the mobile device 2 and the optical positioning system 3 by wireless networking, short-range wireless communication, or wired connection. The mobile device 2 can be a portable electronic device, such as an AR glasses device, an AR headset, a smartphone, a tablet computer, etc., which includes a screen (e.g., lenses of the glasses-type mobile device 2 as shown in FIG. 2) for displaying images, and a camera module (not shown; optional) to capture images from a position of the mobile device 2, and in turn from a position of a user of the mobile device 2. The optical positioning system 3 may adopt, for example, a Polaris Vicra optical tracking system developed by Northern Digital Inc., a Polaris Spectra optical tracking system developed by Northern Digital Inc., an optical tracking system developed by Advanced Realtime Tracking, MicronTracker developed by ClaroNav, etc., but this disclosure is not limited in this respect.

The first embodiment of the surgical navigation method is to be implemented for a surgical operation performed on an operation target 4 which is exemplified as a head (or a brain) of a patient. In step S1, which is performed before the surgical operation, the mobile device 2 stores three-dimensional (3D) imaging information that relates to the operation target 4 in a database (not shown) built in a memory component (e.g., flash memory, a solid-state drive, etc.) thereof. The 3D imaging information may be downloaded from a data source, such as the server 1 or other electronic devices, and originate from Digital Imaging and Communications in Medicine (DICOM) image data, which may be acquired by performing CT, MRI, and/or ultrasound imaging on the operation target 4. The DICOM image data may be native 3D image data or be reconstructed by multiple two-dimensional (2D) sectional images, and relate to blood vessels, nerves, and/or bones. The data source may convert the DICOM image data into files in a 3D image format, such as OBJ and STL formats, by using software (e.g., Amira, developed by Thermo Fisher Scientific), to form the 3D imaging information.

Steps S2 to S5 are performed during the surgical operation. In step S2, the optical positioning system 3 acquires optically-positioned spatial coordinate information (P.D(O) for the mobile device 2, P.T(O) for the operation target 4) relating to the mobile device 2 and the operation target 4 in real time. In step S3, the mobile device constantly obtains a first optically-positioned relative coordinate set (V.TD(O)), which is a vector from the operation target 4 to the mobile device 2, based on the optically-positioned spatial coordinate information (P.D(O), P.T(O)) relating to the mobile device 2 and the operation target 4. In practice, the mobile device 2 may obtain the first optically-positioned relative coordinate set (V.TD(O)) by: (i) the optical positioning system 3 providing the optically-positioned spatial coordinate information (P.D(O), P.T(O)) to the mobile device 2 directly or through the server 1 which is connected to the optical system 3 by wired connection, and the mobile device 2 computing the first optically-positioned relative coordinate set (V.TD(O)) based on the optically-positioned spatial coordinate information (P.D(O), P.T(O)) in real time; or (ii) the optical positioning system 3 providing the optically-positioned spatial coordinate information (P.D(O), P.T(O)) to the server 1 which is connected to the optical system 3 by wired connection, and the server 1 computing the first optically-positioned relative coordinate set (V.TD(O)) based on the optically-positioned spatial coordinate information (P.D(O), P.T(O)) in real time and transmitting the first optically-positioned relative coordinate set (V.TD(O)) to the mobile device 2.

In step S4, the mobile device 2 computes an optically-positioned 3D image that corresponds to the first optically-positioned relative coordinate set (V.TD(O)) based on the 3D imaging information and the first optically-positioned relative coordinate set (V.TD(O)), such that the optically-positioned 3D image presents an image of, for example, the complete brain of the patient as seen from the location of the mobile device 2. Imaging of the optically-positioned 3D image may be realized by software of, for example, Unity (developed by Unity Technologies). Then, the mobile device 2 displays the optically-positioned 3D image based on the first optically-positioned relative coordinate set (V.TD(O)) such that visual perception of the operation target 4 through the mobile device 2 has the optically-positioned 3D image superimposed thereon. In the field of augmented reality (AR), the image superimposition can be realized by various conventional methods, so details thereof are omitted herein for the sake of brevity. It is noted that the surgical optical positioning system 3 used in this embodiment is developed for medical use, thus having high precision of about 0.35 millimeters. Positioning systems that are used for ordinary augmented reality applications do not require such high precision, and may have precision of about only 0.5 meters. Accordingly, the optically-positioned 3D image can be superimposed on the visual perception of the operation target 4 with high precision, so the surgeon and/or the relevant personnel may see a scene where the optically-positioned 3D image is superimposed on the operation target 4 via the mobile device 2. In step S4, the mobile device 2 may further transmit the optically-positioned 3D image to another electronic device (not shown) for displaying the optically-positioned 3D image on another display device 6; or the mobile device 2 may further transmit, to another electronic device, a superimposition 3D image where the optically-positioned 3D image is superimposed on the operation target 4 captured by the camera module of the mobile device 2 so as to display the superimposition 3D image on a display device 6 that is separate from the mobile device 2. Said another electronic device may be the server 1 that is externally coupled to the display device 6, a computer that is externally coupled to the display device 6, or the display device 6 itself. In a case that said another electronic device is the display device 6 itself, the mobile device 2 may use a wireless display technology, such as MiraScreen, to transfer the image to the display device 6 directly.

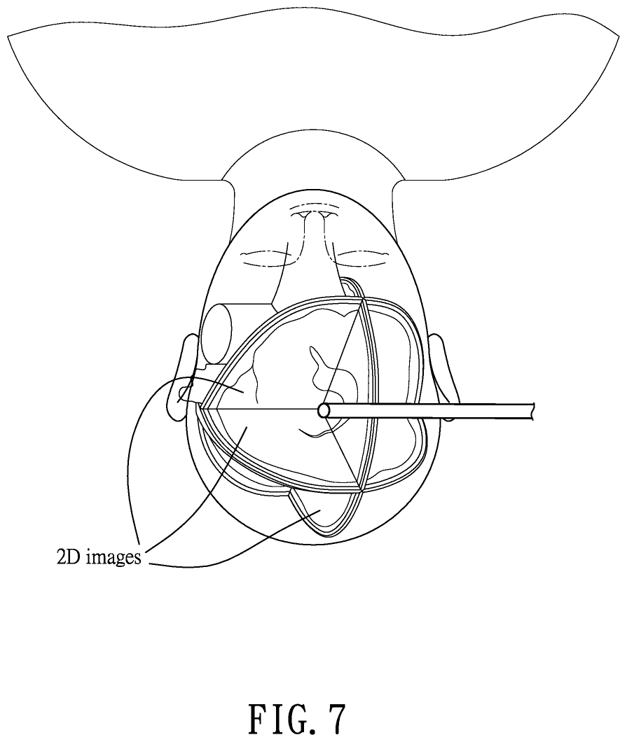

In one implementation, step S1 further includes that the mobile device 2 stores two-dimensional (2D) imaging information that relates to the operation target 4 (e.g., cross-sectional images of the head or brain of the patient) in the database. The 2D imaging information may be downloaded from the data source (e.g., the server 1 or other electronic devices), and originate from DICOM image data. The data source may convert the DICOM image data into files in a 2D image format, such as JPG and NIfTI format, by using DICOM to NIfTI converter software (e.g., dcm2nii, an open source program), to form the 2D imaging information. Step S2 further includes that the optical positioning system 3 acquires optically-positioned spatial coordinate information (P.I(O)) relating to a surgical instrument 5 in real time. Step S3 further includes that the mobile device 2 obtains a second optically-positioned relative coordinate set (V.TI(O)), which is a vector from the operation target 4 to the surgical instrument 5, based on the optically-positioned spatial coordinate information (P.I(O), P.T(O)) relating to the surgical instrument 5 and the operation target 4. In practice, the mobile device 2 may obtain the second optically-positioned relative coordinate set (V.TI(O)) by: (i) the optical positioning system 3 providing the optically-positioned spatial coordinate information (P.I(O), P.T(O)) relating to the surgical instrument 5 and the operation target 4 to the mobile device 2 directly or through the server 1 which is connected to the optical positioning system 3 by wired connection, and the mobile device 2 computing the second optically-positioned relative coordinate set (V.TI(O)) based on the optically-positioned spatial coordinate information (P.I(O), P.T(O)) relating to the surgical instrument 5 and the operation target 4 in real time; or (ii) the optical positioning system providing the optically-positioned spatial coordinate information (P.I(O), P.T(O)) relating to the surgical instrument 5 and the operation target 4 to the server 1 which is connected to the optical system 3 by wired connection, and the server 1 computing the second optically-positioned relative coordinate set (V.TI(O)) based on the optically-positioned spatial coordinate information (P.I(O), P.T(O)) relating to the surgical instrument 5 and the operation target 4 in real time and transmitting the second optically-positioned relative coordinate set (V.TI(O)) to the mobile device 2. Step S4 further includes that the mobile device 2 obtains at least one optically-positioned 2D image (referred to as “the optically-positioned 2D image” hereinafter) that corresponds to the second optically-positioned relative coordinate set (V.TI(O)) based on the 2D imaging information and the second optically-positioned relative coordinate set (V.TI(O)), and displays the optically-positioned 2D image based on the first and second optically-positioned relative coordinate sets (V.TD(O), V.TI(O)) such that visual perception of the operation target 4 through the mobile device 2 has the optically-positioned 2D image superimposed thereon, as exemplified in FIG. 7. The mobile device 2 may obtain the optically-positioned 2D image by (i) computing, based on the 2D imaging information and before the surgical operation, a plurality of 2D candidate images which may possibly be used during the surgical operation, and acquiring, during the surgical operation, at least one of the 2D candidate images to serve as the optically-positioned 2D image based on the second optically-positioned relative coordinate set (V.TI(O)); or (ii) computing, based on the 2D imaging information and the second optically-positioned relative coordinate set (V.TI(O)), the optically-positioned 2D image in real time. In the field of augmented reality, the image superimposition can be realized by various conventional methods, so details thereof are omitted herein for the sake of brevity.

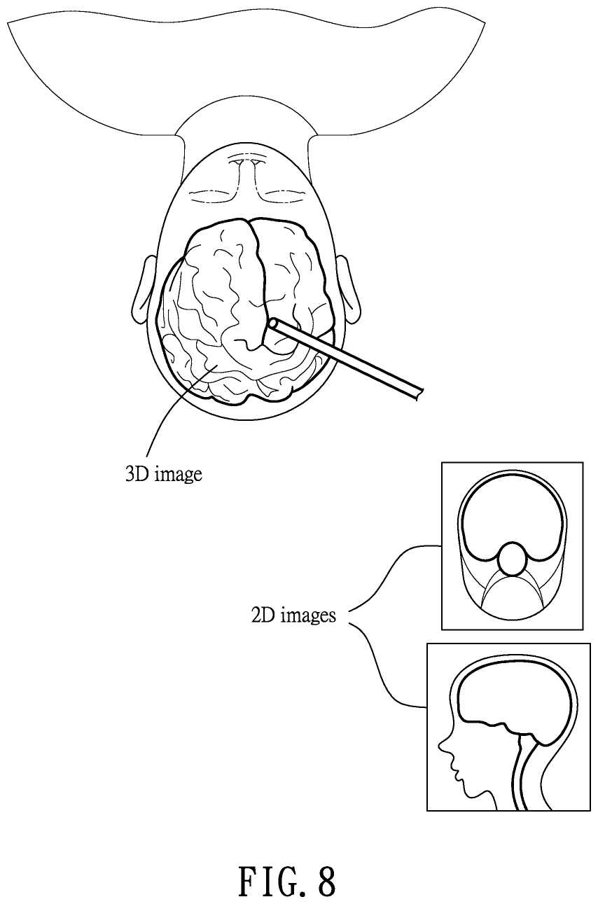

As a result, the surgeon or the relevant personnel can not only see the superimposition 3D image where the optically-positioned 3D image is superimposed on the operation target 4 via the mobile device 2, but can also see the cross-sectional images (i.e., the optically-positioned 2D image) of the operation target 4 corresponding to a position of the surgical instrument 5 (as exemplified in FIG. 8) when the surgical instrument 5 extends into the operation target 4. The mobile device 2 is operable to display one or both of the optically-positioned 3D image and the optically-positioned 2D image, such that visual perception of the operation target 4 through the mobile device 2 has the one or both of the optically-positioned 3D image and the optically-positioned 2D image superimposed thereon. By virtue of the optical positioning system 3 that is capable of providing the optically-positioned spatial coordinate information (P.D(O), P.T(O), P.I(O)) relating to the mobile device 2, the operation target 4 and the surgical instrument 5 with high precision, the mobile device 2 can obtain accurate first and second optically-positioned relative coordinate sets (V.TD(O), V.TI(O)), so that the superimposition of the optically-positioned 2D image and the optically-positioned 3D image on the operation target 4 can have high precision, thereby promoting accuracy and precision of the surgical operation.

It is noted that the 3D imaging information and/or the 2D imaging information may further include information relating to an entry point and a plan (e.g., a surgical route) of the surgical operation for the operation target 4. In such a case, the optically-positioned 3D image and/or the optically-positioned 2D image shows the entry point and the plan of the surgical operation for the operation target 4.

In step S5, the mobile device 2 determines whether an instruction for ending the surgical navigation is received. The flow ends when the determination is affirmative, and goes back to step S2 when otherwise. That is, before receipt of the instruction for ending the surgical navigation, the surgical navigation system 100 continuously repeats steps S2 to S4 to obtain the optically-positioned 3D image and/or the optically-positioned 2D image based on the latest optically-positioned spatial coordinate information (P.D(O), P.T(O) and optionally P.I(O))), so the scenes where the optically-positioned 3D image and/or the optically-positioned 2D image is superimposed on the operation target 4 as seen by the surgeon and/or the relevant personnel through the mobile device 2 are constantly updated in real time in accordance with movement of the surgeon and/or the relevant personnel, and the mobile device 2 can provide information relating to internal structure of the operation target 4 to the surgeon and/or the relevant personnel in real time, thereby assisting the surgeon and/or the relevant personnel in making decisions during the surgical operation.

Furthermore, in step S4, the mobile device 2 may further transmit the optically-positioned 3D image and/or the optically-positioned 2D image to another electronic device for displaying the optically-positioned 3D image on another display device 6; or the mobile device 2 may further transmit, to another electronic device, a superimposition 3D/2D image where the optically-positioned 3D image and/or the optically-positioned 2D image is superimposed on the operation target 4 captured by the camera module of the mobile device 2, so as to display the superimposition 3D image on the display device 6 separate from the mobile device 2. Said another electronic device may be the server 1 that is externally coupled to the display device 6, a computer that is externally coupled to the display device 6, and the display device 6 itself. In a case that said another electronic device is the display device 6 itself, the mobile device 2 may use a wireless display technology to transfer the image(s) to the display device 6 directly. As a result, persons other than the surgeon and the relevant personnel may experience the surgical operation by seeing the images of the surgical operation from the perspective of the surgeon (or the relevant personnel) via the display device 6, which is suitable for education purposes.

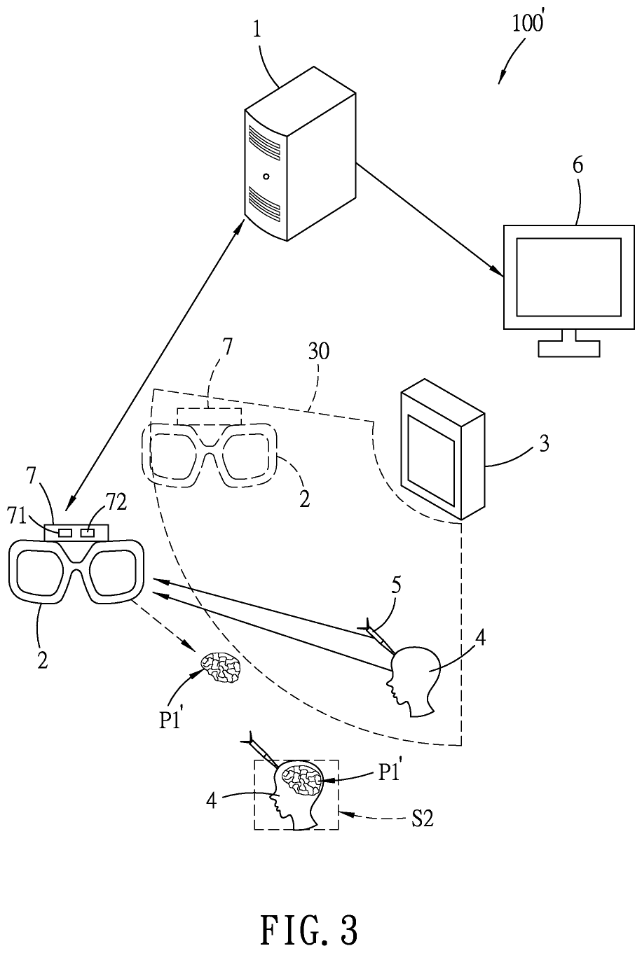

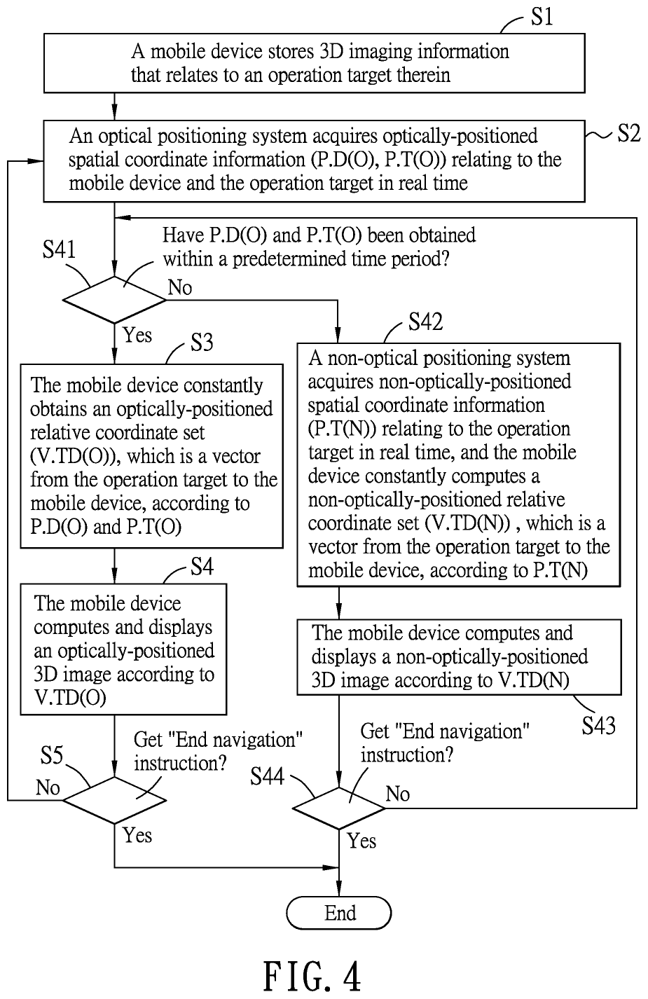

Referring to FIG. 3, when the mobile device 2 is not located within the limited positioning range 30 of the optical positioning system 3 or when the optical positioning system 3 is out of order, the optically-positioning spatial coordinate information becomes unavailable. In order to solve such a problem, FIG. 3 illustrates that the second embodiment of the surgical navigation method according to this disclosure is implemented by a surgical navigation system 100′ that, in comparison to the surgical navigation system 100 as shown in FIG. 2, further includes a non-optical positioning system 7 mounted to and communicatively coupled to the mobile device 2. Further referring to FIG. 4, the flow for the second embodiment further includes steps S41-S44. In step S41, which is performed between steps S2 and S3, the mobile device 2 determines whether the mobile device 2 has acquired the first optically-positioned spatial coordinate information (P.D(O), P,T(O)) within a predetermined time period from the last receipt of the first optically-positioned spatial coordinate information (P.D(O), P,T(O)). The flow continues to step S4 when it is determined that the mobile device 2 has acquired the first optically-positioned spatial coordinate information (P.D(O), P,T(O)) within the predetermined time period, and goes to step S42 when otherwise (i.e., when the mobile device 2 fails to acquire the first optically-positioned spatial coordinate information (P.D(O), P,T(O)) within the predetermined time period). In step S42, the non-optical positioning system 7 acquires non-optically-positioned spatial coordinate information (P.T(N)) relating to the operation target 4 in real time, and the mobile device 2 constantly computes a first non-optically-positioned relative coordinate set (V.TD(N)), which is a vector from the operation target 4 to the mobile device 2, based on the non-optically-positioned spatial coordinate information (P.T(N)) in real time. In this embodiment, the non-optical positioning system 7 may be an image positioning system 71, a gyroscope positioning system 72, or a combination of the two. The image positioning system 71 may be realized by, for example, a Vuforia AR platform, and the gyroscope positioning system 72 may be built in or externally mounted to the mobile device 2. The gyroscope positioning system 72 may position the mobile device 2 with respect to, for example, the operation target 4, with reference to the optically-positioned spatial coordinate information (P.D(O), P.T(O)) that is previously obtained by the optical positioning system 3.

In step S43, the mobile device 2 computes a non-optically-positioned 3D image that corresponds to the first non-optically-positioned relative coordinate set (V.TD(N)) based on the 3D imaging information and the first non-optically-positioned relative coordinate set (V.TD(N)), and displays the non-optically-positioned 3D image based on the first non-optically-positioned relative coordinate set (V.TD(N)) such that visual perception of the operation target 4 through the mobile device 2 has the non-optically-positioned 3D image superimposed thereon. In step S44, the mobile device 2 determines whether the instruction for ending the surgical navigation is received. The flow ends when the determination is affirmative, and goes back to step S41 when otherwise. Accordingly, when the mobile device 2 is not within the positioning range 30 of the optical positioning system 3 or when the optical positioning system 3 is out of order, the surgeon and/or the relevant personnel can still utilize the surgical navigation. In practice, the non-optical positioning system 7 may also be used alone in the surgical navigation system, although it has lower positioning precision when compared with the optical positioning system 7.

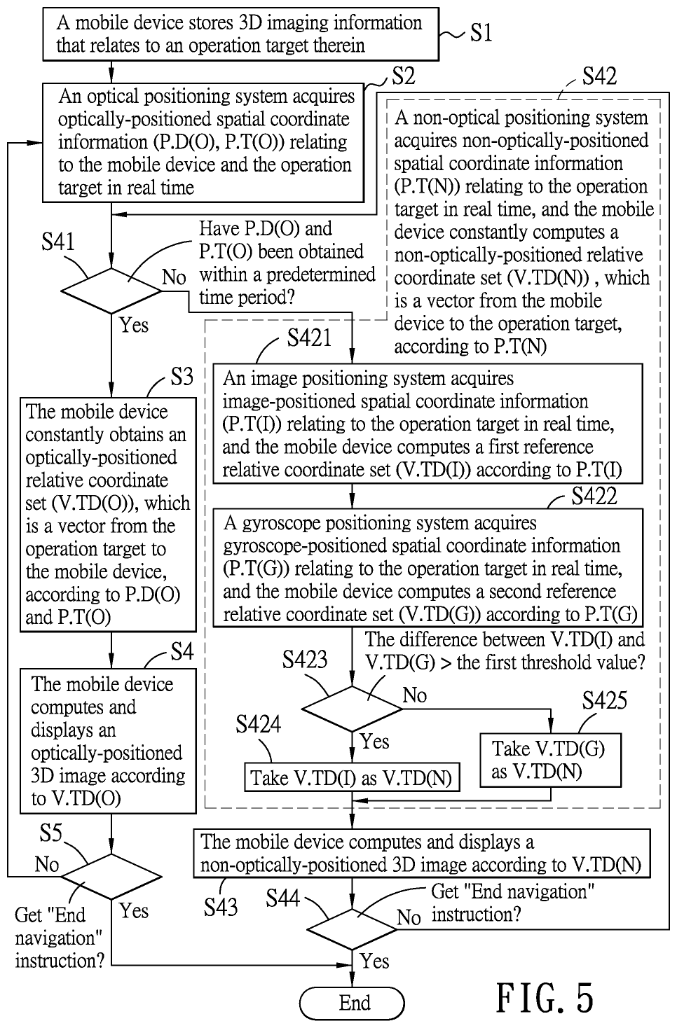

In this embodiment, the non-optical positioning system 7 includes both of the image positioning system 71 and the gyroscope positioning system 72, and step S42 includes sub-steps S421-S425 (see FIG. 5). In sub-step S421, the mobile device 2 causes the image positioning system 71 to acquire image-positioned spatial coordinate information relating to the operation target 4, and the mobile device 2 computes a first reference relative coordinate set (V.TD(I)), which is a vector from the operation target 4 to the mobile device 2, based on the image-positioned spatial coordinate information in real time.

In step S422, the mobile device 2 causes the gyroscope positioning system 72 to acquire gyroscope-positioned spatial coordinate information relating to the operation target 4, and the mobile device 2 computes a second reference relative coordinate set (V.TD(G)), which is a vector from the operation target 4 to the mobile device 2, based on the gyroscope-positioned spatial coordinate information in real time. The non-optically-positioned spatial coordinate information (P.T(N)) includes the image-positioned spatial coordinate information and the gyroscope-positioned spatial coordinate information.

In step S423, the mobile device 2 determines whether a difference between the first and second reference relative coordinate sets (V.TD(I), V.TD(G)) is greater than a first threshold value. The flow goes to step S424 when the determination is affirmative, and goes to step S425 when otherwise.

In step S424, the mobile device 2 takes the first reference relative coordinate set (V.TD(I)) as the first non-optically-positioned relative coordinate set (V.TD(N)). In step S425, the mobile device 2 takes the second reference relative coordinate set (V.TD(G)) as the first non-optically-positioned relative coordinate set (V.TD(N)). Generally, the image positioning system 71 has higher precision than the gyroscope positioning system 72. However, because a speed of the gyroscope positioning system 72 acquiring the gyroscope-positioned spatial coordinate information is faster than a speed of the image positioning system 71 acquiring the image-positioned spatial coordinate information, the second reference relative coordinate set (V.TD(G)) has higher priority in serving as the first non-optically-positioned relative coordinate set (V.TD(N)), unless the difference between the first and second reference relative coordinate sets (V.TD(I), V.TD(G)) is greater than the first threshold value.

In the implementation where the mobile device 2 further stores the 2D imaging information in step S1, step S42 further includes that the non-optical positioning system 7 acquires non-optically-positioned spatial coordinate information (P.I(N)) relating to the surgical instrument 5 in real time, and the mobile device 2 obtains a second non-optically-positioned relative coordinate set (V.TI(N)), which is a vector from the operation target 4 to the surgical instrument 5, based on the non-optically-positioned spatial coordinate information (P.I(N), P.T(N)) relating to the surgical instrument 5 and the operation target 4. Step S43 further includes that the mobile device 2 obtains at least one non-optically-positioned 2D image (referred as “the non-optically-positioned 2D image” hereinafter) corresponding to the second non-optically-positioned relative coordinate set (V.TI(N)) based on the 2D imaging information and the second non-optically-positioned relative coordinate set (V.TI(N)), and displays the non-optically-positioned 2D image based on the first non-optically-positioned relative coordinate set (V.TD(N)) and the second non-optically-positioned relative coordinate set (V.TI(N)) such that visual perception of the operation target 4 through the mobile device 2 has the non-optically-positioned 2D image superimposed thereon. Method for obtaining the non-optically-positioned 2D image is similar to that for obtaining the optically-positioned 2D image, so details thereof are omitted herein for the sake of brevity. Before receipt of the instruction for ending the surgical navigation, the flow goes back to step S41 after step S44. If the mobile device 2 still fails to acquire the first optically-positioned spatial coordinate information (P.D(O), P,T(O)) within the predetermined time period in step S41, steps S42 to S44 are repeated, so as to continuously obtain the non-optically-positioned 3D image and/or the non-optically-positioned 2D image based on the latest non-optically-positioned spatial coordinate information (P.T(N), optionally P.I(N)), so the scenes where the non-optically-positioned 3D image and/or the non-optically-positioned 2D image is superimposed on the operation target 4 as seen by the surgeon and/or the relevant personnel through the mobile device 2 are constantly updated in real time in accordance with movement of the surgeon and/or the relevant personnel, and the mobile device can provide information relating to internal structure of the operation target 4 to the surgeon and/or the relevant personnel in real time, thereby assisting the surgeon and/or the relevant personnel in making decisions during the surgical operation.

Furthermore, since the non-optical positioning system 7 of this embodiment includes both of the image positioning system 71 and the gyroscope positioning system 72, in the implementation where the mobile device 2 further stores the 2D imaging information in step S1, step S421 further includes that the mobile device 2 causes the image positioning system 71 to acquire image-positioned spatial coordinate information relating to the surgical instrument 5, and the mobile device 2 computes a third reference relative coordinate set (V.TI(I)), which is a vector from the operation target 4 to the surgical instrument 5, based on the image-positioned spatial coordinate information relating to the surgical instrument 5 and the operation target 4 in real time; and step S422 further includes that the mobile device 2 causes the gyroscope positioning system 72 to acquire gyroscope-positioned spatial coordinate information relating to the surgical instrument 5, and the mobile device 2 computes a fourth reference relative coordinate set (V.TI(G)), which is a vector from the operation target 4 to the surgical instrument 5, based on the gyroscope-positioned spatial coordinate information relating to the surgical instrument 5 and the operation target 4 in real time. Then, the mobile device 2 determines whether a difference between the third and fourth reference relative coordinate sets (V.TI(I), V.TI(G)) is greater than a second threshold value. The mobile device 2 takes the third reference relative coordinate sets (V.TI(I)) as the second non-optically-positioned relative coordinate set (V.TI(N)) when the determination is affirmative, and takes the fourth reference relative coordinate sets (V.TI(G)) as the second non-optically-positioned relative coordinate set (V.TI(N)) when otherwise.

In practice, since the optical positioning system 3 may need to first transmit the optically-positioned spatial coordinate information (P.D(O), P.T(O), optionally P.I(O)) to the server 1 through wired connection, and then the server 1 provides the optically-positioned spatial coordinate information (P.D(O), P.T(O), optionally P.I(O)) or the first optically-positioned relative coordinate set to the mobile device 2, transmission delay may exist. A serious transmission delay may lead to significant difference between the computed first optically-positioned relative coordinate set and a current coordinate, which is a vector from the operation target 4 to the mobile device 2, so the first optically-positioned 3D image may not be accurately superimposed on the operation target 4 in terms of visual perception, causing image jiggling. On the other hand, the non-optical positioning system 7 that is mounted to the mobile device 2 transmits the non-optically positioned spatial coordinate information to the mobile device 2 directly, so the transmission delay may be significantly reduced, alleviating image jiggling.

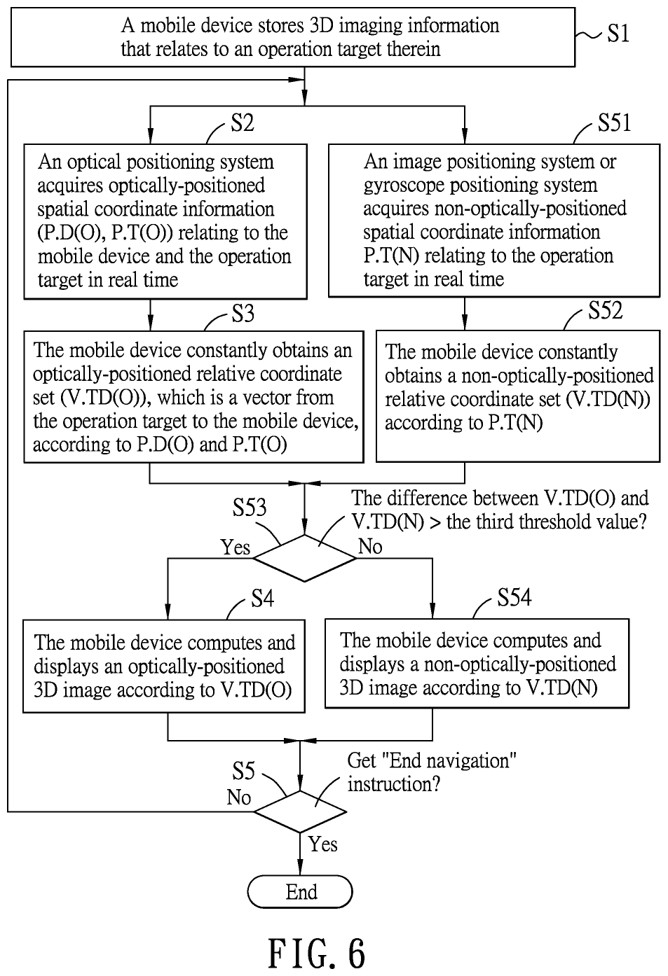

Accordingly, the third embodiment of the surgical navigation method according to this disclosure is proposed to be implemented by the surgical navigation system 100′ as shown in FIG. 3, and has a flow as shown in FIG. 6.

In this embodiment, steps S1-S5 are the same as those of the first embodiment. While the optical positioning system 3 acquires the optically-positioned spatial coordinate information (P.D(O), P.T(O)) relating to the mobile device 2 and the operation target 4 in real time (step S2), the image positioning system 71 or the gyroscope positioning system 72 of the non-optical positioning system 7 also acquires the non-optically-positioned spatial coordinate information (P.T(N)) relating to the operation target 4 (step S51). While the mobile device 2 obtains the first optically-positioned relative coordinate set (V.TD(O)) in real time (step S3), the mobile device 2 also constantly computes the first non-optically-positioned relative coordinate set (V.TD(N)) based on the non-optically-positioned spatial coordinate information (P.T(N)) relating to the operation target 4 in real time (step S52).

In step S53, the mobile device 2 determines whether a difference between the first optically-positioned relative coordinate set (V.TD(O)) and the first non-optically-positioned relative coordinate set (V.TD(N)) is greater than a third threshold value. The flow goes to step S4 when the determination is affirmative, and goes to step S54 when otherwise.

In step S54, the mobile device 2 computes the non-optically-positioned 3D image that corresponds to the first non-optically-positioned relative coordinate set (V.TD(N)) based on the 3D imaging information and the first non-optically-positioned relative coordinate set (V.TD(N)), and displays the non-optically-positioned 3D image based on the first non-optically-positioned relative coordinate set (V.TD(N)) such that visual perception of the operation target 4 through the mobile device 2 has the non-optically-positioned 3D image superimposed thereon.

In the implementation where the mobile device 2 further stores the 2D imaging information in step S1, step S51 further includes that the image positioning system 71 or the gyroscope positioning system 72 of the non-optical positioning system 7 acquires the non-optically-positioned spatial coordinate information (P.I(N)) relating to the surgical instrument 5 in real time; and step S52 further includes that the mobile device 2 computes the second non-optically-positioned relative coordinate set (V.TI(N)) based on the non-optically-positioned spatial coordinate information (P.I(N), P.T(N)) relating to the surgical instrument 5 and the operation target 4 in real time. Then, the mobile device 2 determines whether a difference between the second optically-positioned relative coordinate set (V.TI(O)) and the second non-optically-positioned relative coordinate set (V.TI(N)) is greater than a fourth threshold value. The mobile device 2 obtains the optically-positioned 2D image based on the second optically-positioned relative coordinate set (V.TI(O)), and displays the optically-positioned 2D image based on the first optically-positioned relative coordinate set (V.TD(O)) (or the first non-optically-positioned relative coordinate set (V.TD(N))) and the second optically-positioned relative coordinate set (V.TI(O)) when the determination is affirmative, such that visual perception of the operation target 4 through the mobile device 2 has the optically-positioned 2D image superimposed thereon; and the mobile device 2 obtains the non-optically-positioned 2D image based on the second non-optically-positioned relative coordinate set (V.TI(N)), and displays the non-optically-positioned 2D image based on the first optically-positioned relative coordinate set (V.TD(O)) (or the first non-optically-positioned relative coordinate set (V.TD(N))) and the second non-optically-positioned relative coordinate set (V.TI(N)) when otherwise, such that visual perception of the operation target 4 through the mobile device 2 has the non-optically-positioned 2D image superimposed thereon. In other words, the third embodiment primarily uses the non-optical positioned system 7 for obtaining the relative coordinate set(s) in order to avoid image jiggling, unless a positioning error of the non-optical positioned system 7 is too large (note that the optical positioned system 3 has higher precision in positioning).

In summary, the embodiments of this disclosure include the optical positioning system 3 acquiring the optically-positioned spatial coordinate information (P.D(O), P.T(O), P.I(O)) relating to the mobile device 2, the operation target 4 and the surgical instrument 5, thereby achieving high precision in positioning, so that the mobile device can superimpose the optically-positioned 3D/2D image(s) on the operation target 4 for visual perception with high precision at a level suitable for medical use, promoting the accuracy and precision of the surgical operation. In the second embodiment, when the mobile device 2 is not within the positioning range 30 of the optical positioning system 3 or when the optical positioning system 3 is out of order, the mobile device 2 can still cooperate with the non-optical positioning system 7 to obtain the non-optically-positioned 3D/2D image(s) and superimpose the non-optically-positioned 3D/2D image(s) on the operation target 4 for visual perception, so that the surgical navigation is not interrupted. In the third embodiment, by appropriately switching use of information from the optical positioning system 3 and the non-optical positioning system 7, possible image jiggling may be alleviated.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects, and that one or more features or specific details from one embodiment may be practiced together with one or more features or specific details from another embodiment, where appropriate, in the practice of the disclosure.

While the disclosure has been described in connection with what is (are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A surgical navigation method, comprising, before a surgical operation is performed on an operation target:
(A) by a mobile device that is capable of computation and displaying images, storing three-dimensional (3D) imaging information that relates to the operation target therein;
the surgical navigation method comprising, during the surgical operation:
(B) by an optical positioning system, acquiring first optically-positioned spatial coordinate information relating to the mobile device and the operation target in real time;
(C) by the mobile device, obtaining a first optically-positioned relative coordinate set, which is a vector from the operation target to the mobile device, based on the first optically-positioned spatial coordinate information acquired in step (B); and
(D) by the mobile device, computing an optically-positioned 3D image that corresponds to the first optically-positioned relative coordinate set based on the 3D imaging information and the first optically-positioned relative coordinate set, and displaying the optically-positioned 3D image based on the first optically-positioned relative coordinate set such that visual perception of the operation target through the mobile device has the optically-positioned 3D image superimposed thereon.

2. The surgical navigation method of claim 1, wherein step (B) further includes: by the optical positioning system, transmitting the first optically-positioned spatial coordinate information to the mobile device; and, in step (C), the mobile device performs computation based on the first optically-positioned spatial coordinate information to obtain the first optically-positioned relative coordinate set in real time.

3. The surgical navigation method of claim 1, wherein step (B) further includes: by the optical positioning system, transmitting the first optically-positioned spatial coordinate information to a server which is connected to the optical positioning system by wired connection; and said surgical navigation method further comprises: by the server, computing the first optically-positioned relative coordinate set based on the first optically-positioned spatial coordinate information in real time, and transmitting the first optically-positioned relative coordinate set to the mobile device.

4. The surgical navigation method of claim 1, wherein step (A) further includes: by the mobile device, storing two-dimensional (2D) imaging information that relates to the operation target therein; step (B) further includes: by the optical positioning system, acquiring second optically-positioned spatial coordinate information that relates to a surgical instrument in real time; step (C) further includes: by the mobile device, obtaining a second optically-positioned relative coordinate set, which is a vector from the operation target to the surgical instrument, based on third optically-positioned spatial coordinate information that relates to the surgical instrument and the operation target; and step (D) further includes: by the mobile device, obtaining at least one optically-positioned 2D image corresponding to the second optically-positioned relative coordinate set based on the 2D imaging information and the second optically-positioned relative coordinate set, and displaying the at least one optically-positioned 2D image according to the first optically-positioned relative coordinate set and the second optically-positioned relative coordinate set such that visual perception of the operation target through the mobile device has the at least one optically-positioned 2D image superimposed thereon.

5. The surgical navigation method of claim 4, wherein step (B) further includes: by the optical positioning system, transmitting the third optically-positioned spatial coordinate information to the mobile device; and, in step (C), the mobile device performs computation based on the third optically-positioned spatial coordinate information to obtain the second optically-positioned relative coordinate set in real time.

6. The surgical navigation method of claim 4, wherein step (B) further includes: by the optical positioning system, transmitting the third optically-positioned spatial coordinate information to a server which is connected to the optical positioning system by wired connection; and said surgical navigation method further comprises: by the server, computing the second optically-positioned relative coordinate set based on the third optically-positioned spatial coordinate information in real time, and transmitting the second optically-positioned relative coordinate set to the mobile device.

7. The surgical navigation method of claim 4, further comprising, after step (A) and before the surgical operation is performed: by the mobile device, computing, based on the 2D imaging information, a plurality of 2D candidate images which will possibly be used during the surgical operation, and wherein step (D) further includes: by the mobile device, acquiring at least one of the 2D candidate images to serve as the at least one optically-positioned 2D image based on the second optically-positioned relative coordinate set.

8. The surgical navigation method of claim 4, wherein step (D) further includes: by the mobile device, computing, based on the 2D imaging information and the second optically-positioned relative coordinate set, the at least one optically-positioned 2D image in real time.

9. The surgical navigation method of claim 4, wherein, step (D) further includes: by the mobile device, transmitting at least one of the optically-positioned 3D image or the at least one optically-positioned 2D image to an electronic device for displaying the at least one of the optically-positioned 3D image or the at least one optically-positioned 2D image on a display device other than the mobile device, where the electronic device is a server that is externally coupled to the display device, a computer that is externally coupled to the display device, or the display device itself.

10. The surgical navigation method of claim 4, wherein the mobile device includes a camera module to capture images from a position of the mobile device, and step (D) further includes: by the mobile device, transmitting a superimposition 3D image where the optically-positioned 3D image is superimposed on the operation target captured by the camera module of the mobile device to an electronic device for displaying the superimposition 3D image on a display device other than the mobile device, where the electronic device is a server that is externally coupled to the display device, a computer that is externally coupled to the display device, or the display device itself.

11. The surgical navigation method of claim 4, wherein at least one of the 3D imaging information or the 2D imaging information includes information relating to an entry point and a plan of the surgical operation to be performed on the operation target; and at least one of the optically-positioned 3D image or the at least one optically-positioned 2D image shows the entry point and the plan of the surgical operation.

12. The surgical navigation method of claim 1, wherein step (D) further includes: by the mobile device, transmitting the optically-positioned 3D image to an electronic device for displaying the optically-positioned 3D image on a display device other than the mobile device, where the electronic device is a server that is externally coupled to the display device, a computer that is externally coupled to the display device, or the display device itself.

13. The surgical navigation method of claim 1, wherein the mobile device is mounted with and communicatively coupled to a non-optical positioning system, and said surgical navigation method further comprises, when the mobile device fails to acquire the first optically-positioned spatial coordinate information within a predetermined time period in step (B) during the surgical operation:
(E) by the non-optical positioning system, acquiring first non-optically-positioned spatial coordinate information relating to the operation target in real time;
(F) by the mobile device, computing a first non-optically-positioned relative coordinate set, which is a vector from the operation target to the mobile device, based on the first non-optically-positioned spatial coordinate information in real time; and
(G) by the mobile device, computing a non-optically-positioned 3D image that corresponds to the first non-optically-positioned relative coordinate set based on the 3D imaging information and the first non-optically-positioned relative coordinate set, and displaying the non-optically-positioned 3D image based on the first non-optically-positioned relative coordinate set such that visual perception of the operation target through the mobile device has the non-optically-positioned 3D image superimposed thereon.

14. The surgical navigation method of claim 13, wherein step (A) further includes: by the mobile device, storing two-dimensional (2D) imaging information that relates to the operation target therein; step (E) further includes: by the non-optical positioning system, acquiring second non-optically-positioned spatial coordinate information relating to a surgical instrument in real time; step (F) further includes: by the mobile device, obtaining a second non-optically-positioned relative coordinate set, which is a vector from the operation target to the surgical instrument, based on third non-optically-positioned spatial coordinate information, which is the non-optically-positioned spatial coordinate information relating to the surgical instrument and the operation target; and step (G) further includes: by the mobile device, obtaining at least one non-optically-positioned 2D image corresponding to the second non-optically-positioned relative coordinate set based on the 2D imaging information and the second non-optically-positioned relative coordinate set, and displaying the at least one non-optically-positioned 2D image based on the first non-optically-positioned relative coordinate set and the second non-optically-positioned relative coordinate set such that visual perception of the operation target through the mobile device has the at least one non-optically-positioned 2D image superimposed thereon.

15. The surgical navigation method of claim 13, wherein the non-optical positioning system includes an image positioning system and a gyroscope positioning system;
wherein step (E) includes: by the image positioning system, acquiring image-positioned spatial coordinate information relating to the operation target; and, by the gyroscope positioning system, acquiring gyroscope-positioned spatial coordinate information relating to the operation target, the first non-optically-positioned spatial coordinate information including the image-positioned spatial coordinate information and the gyroscope-positioned spatial coordinate information; and
wherein step (F) includes:
by the mobile device, computing a first reference relative coordinate set, which is a vector from the operation target to the mobile device, based on the image-positioned spatial coordinate information in real time, and computing a second reference relative coordinate set, which is a vector from the operation target to the mobile device, based on the gyroscope-positioned spatial coordinate information in real time; and
by the mobile device, taking the first reference relative coordinate set as the first non-optically-positioned relative coordinate set upon determining that a difference between the first and second reference relative coordinate sets is greater than a first threshold value, and taking the second reference relative coordinate set as the first non-optically-positioned relative coordinate set upon determining that the difference between the first and second reference relative coordinate sets is not greater than the first threshold value.

16. The surgical navigation method of claim 15, wherein step (A) further includes: by the mobile device, storing two-dimensional (2D) imaging information that relates to the operation target therein;
wherein step (E) further includes: by the image positioning system, acquiring image-positioned spatial coordinate information relating to a surgical instrument; and, by the gyroscope positioning system, acquiring gyroscope-positioned spatial coordinate information relating to the surgical instrument;
wherein step (F) further includes:
by the mobile device, computing a third reference relative coordinate set, which is a vector from the operation target to the surgical instrument, based on the image-positioned spatial coordinate information related to the surgical instrument and the operation target in real time, and computing a fourth reference relative coordinate set, which is a vector from the operation target to the surgical instrument, based on the gyroscope-positioned spatial coordinate information relating to the surgical instrument and the operation target in real time; and
by the mobile device, taking the third reference relative coordinate set as a second non-optically-positioned relative coordinate set upon determining that a difference between the third and fourth reference relative coordinate sets is greater than a second threshold value, and taking the fourth reference relative coordinate set as the second non-optically-positioned relative coordinate set upon determining that the difference between the third and fourth reference relative coordinate sets is not greater than the second threshold value; and
wherein step (G) further includes: by the mobile device, obtaining at least one non-optically-positioned 2D image corresponding to the second non-optically-positioned relative coordinate set based on the 2D imaging information and the second non-optically-positioned relative coordinate set, and displaying the at least one non-optically-positioned 2D image based on the first non-optically-positioned relative coordinate set and the second non-optically-positioned relative coordinate set such that visual perception of the operation target through the mobile device has the at least one non-optically-positioned 2D image superimposed thereon.

17. The surgical navigation method of claim 1, wherein the mobile device is mounted with and communicatively coupled to a non-optical positioning system, and said surgical navigation method further comprises:
(E) by the non-optical positioning system, acquiring non-optically-positioned spatial coordinate information relating to the operation target in real time;
wherein step (C) further includes: by the mobile device, computing a first non-optically-positioned relative coordinate set, which is a vector from the operation target to the mobile device, based on the non-optically-positioned spatial coordinate information in real time; and
wherein said surgical navigation method further comprises: (F) by the mobile device, determining whether a difference between the first optically-positioned relative coordinate set and the first non-optically-positioned relative coordinate set is greater than a first threshold value; and
wherein, in step (D), the step of computing an optically-positioned 3D image and displaying the optically-positioned 3D image is performed when the determination made in step (F) is affirmative, and step (D) further includes: by the mobile device when the determination made in step (F) is negative, computing a non-optically-positioned 3D image that corresponds to the first non-optically-positioned relative coordinate set based on the 3D imaging information and the first non-optically-positioned relative coordinate set, and displaying the non-optically-positioned 3D image based on the first non-optically-positioned relative coordinate set such that visual perception of the operation target through the mobile device has the non-optically-positioned 3D image superimposed thereon.

18. The surgical navigation method of claim 17, wherein step (A) further includes: by the mobile device, storing two-dimensional (2D) imaging information that relates to the operation target therein;
wherein step (B) further includes: by the optical positioning system, acquiring second optically-positioned spatial coordinate information relating to a surgical instrument in real time;
wherein step (E) further includes: by the non-optical positioning system, acquiring non-optically-positioned spatial coordinate information relating to the surgical instrument in real time;
wherein step (C) further includes: by the mobile device, computing a second optically-positioned relative coordinate set, which is a vector from the operation target to the surgical instrument, based on third optically-positioned spatial coordinate information relating to the surgical instrument and the operation target in real time; and computing a second non-optically-positioned relative coordinate set, which is a vector from the operation target to the surgical instrument, based on the non-optically-positioned spatial coordinate information relating to the surgical instrument and the operation target in real time;
wherein said surgical navigation method further comprises: (G) by the mobile device, determining whether a difference between the second optically-positioned relative coordinate set and the second non-optically-positioned relative coordinate set is greater than a second threshold value; and
wherein step (D) further includes:
by the mobile device when the determination made in step (G) is affirmative, obtaining at least one optically-positioned 2D image corresponding to the second optically-positioned relative coordinate set based on the 2D imaging information and the second optically-positioned relative coordinate set, and displaying the at least one optically-positioned 2D image based on the first optically-positioned relative coordinate set and the second optically-positioned relative coordinate set such that visual perception of the operation target through the mobile device has the at least one optically-positioned 2D image superimposed thereon; and
by the mobile device when the determination made in step (G) is negative, obtaining at least one non-optically-positioned 2D image corresponding to the second non-optically-positioned relative coordinate set based on the 2D imaging information and the second non-optically-positioned relative coordinate set, and displaying the at least one non-optically-positioned 2D image based on the first non-optically-positioned relative coordinate set and the second non-optically-positioned relative coordinate set such that visual perception of the operation target through the mobile device has the at least one non-optically-positioned 2D image superimposed thereon.

19. A surgical navigation system, comprising a mobile device that is capable of computation and displaying images, and an optical positioning system that cooperates with said mobile device to perform:
before a surgical operation is performed on an operation target:
(A) by said mobile device, storing three-dimensional (3D) imaging information that relates to the operation target therein; and
during the surgical operation:
(B) by said optical positioning system, acquiring first optically-positioned spatial coordinate information relating to said mobile device and the operation target in real time;
(C) by said mobile device, obtaining a first optically-positioned relative coordinate set, which is a vector from the operation target to said mobile device, based on the first optically-positioned spatial coordinate information acquired by said optical positioning system; and
(D) by said mobile device, computing an optically-positioned 3D image that corresponds to the first optically-positioned relative coordinate set based on the 3D imaging information and the first optically-positioned relative coordinate set, and displaying the optically-positioned 3D image based on the first optically-positioned relative coordinate set such that visual perception of the operation target through said mobile device has the optically-positioned 3D image superimposed thereon.

20. The surgical navigation system of claim 19, further comprising a non-optical positioning system that cooperates with said mobile device and said optical positioning system to perform, when said mobile device fails to obtain the first optically-positioned spatial coordinate information within a predetermined time period during the surgical operation:
(E) by said non-optical positioning system, acquiring non-optically-positioned spatial coordinate information relating to the operation target in real time;
(F) by said mobile device, computing a first non-optically-positioned relative coordinate set, which is a vector from the operation target to said mobile device, based on the non-optically-positioned spatial coordinate information in real time; and
(G) by said mobile device, computing a non-optically-positioned 3D image that corresponds to the first non-optically-positioned relative coordinate set based on the 3D imaging information and the first non-optically-positioned relative coordinate set, and displaying the non-optically-positioned 3D image based on the first non-optically-positioned relative coordinate set such that visual perception of the operation target through said mobile device has the non-optically-positioned 3D image superimposed thereon.
